(19) United States
(12) Patent Application Publication
(10) Pub. No.: US 2016/0025702 A1
(43) Pub. Date: Jan. 28, 2016

(54) SYSTEMS, DEVICES AND METHODS FOR TRANSLOCATION CONTROL

(71) Applicant: ARIZONA BOARD OF REGENTS on behalf of Arizona STATE UNIVERSITY, Scottsdale, AZ (US)

(72) Inventors: Stuart LINDSAY, Phoenix, AZ (US); Brett GYARFAS, Chandler, AZ (US); Predrag KRSTIC, Scottsdale, AZ (US); Padmini KRISHNAKUMAR, Scottsdale, AZ (US)

(21) Appl. No.: 14/775,360

(22) PCT Filed: Mar. 12, 2014
(86) PCT No.: PCT/US14/24630

§ 371 Date: Sep. 11, 2015

Related U.S. Application Data
(60) Provisional application No. 61/780,477, filed on Mar. 13, 2013.

(51) Int. Cl. G01N 33/487

(52) U.S. Cl. CPC G01N 33/48721

(57) ABSTRACT

Some embodiments of the present disclosure are directed to systems, methods and devices for controlling the transit of a molecule across a nanopore. Some embodiments are directed to a device comprising a first compartment, a second compartment, a first pair of electrodes comprising a first electrode provided in the first compartment and a second electrode provided in the second compartment, a partition separating the first compartment from the second compartment, an orifice provided in the partition, a second pair of electrodes arranged proximate the orifice, the second pair of electrodes being functionalized with molecules, and a tunnel gap comprising the spacing between the second pair of electrodes.

### RELATED APPLICATIONS

This application claims benefit under 35 U.S.C. §119(e) of U.S. provisional patent application No. 61/780,477, entitled, “SYSTEMS, DEVICES AND METHODS FOR TRANSLOCATION CONTROL”, filed on Mar. 13, 2013, the entire disclosure of which is herein incorporated by reference.

Embodiments of this disclosure were made with government support under NIH Grant No. R01 HG006323, awarded by the National Institute of Health. The U.S. Government has certain rights in inventions disclosed herein.

### BACKGROUND OF THE DISCLOSURE

It has been widely recognized that a major challenge to identifying and sequencing of polymers in a nanopore is the rapid speed of highly charged molecules driven through the pore by electrophoresis.¹ At a voltage large enough to overcome thermal fluctuations² (i.e., several times thermal energy or voltages that are several times 25 mV), the translocation speed is on the order of microseconds (or less) per DNA base, in the case of double stranded DNA. Current schemes for slowing translocation have been proposed. In the first, a molecular motor that processes DNA is used to trap a DNA molecule as it is drawn into a nanopore (FIG. 1). DNA 3 terminated in a modified strand 4 that blocks polymerization is captured by a DNA polymerase 2 that cannot process it because of strand 4. An unhybridized region 5 hangs out of the polymerase and is drawn into the nanopore 1 by electrophoresis. With an adequate electric driving force, the blocking strand 4 is peeled from the DNA as it is pulled into the nanopore. Once the blocking strand is removed, synthesis of the complementary strand commences in the presence of nucleotides. This results in a relatively slow pulling of the overhanging strand 5 back through the pore, allowing sequence to be read.³ This scheme is restricted to DNA sequencing.

In the second approach, a solid-state translocation device is provided called the DNA transistor^{4,5} (FIG. 2). The DNA molecule (or other charged polymer) 13 is drawn into a solid state nanopore 10 where a set of three embedded electrodes (separated by dielectric 12) apply opposing electric fields. If the fields are large enough, the motion of the DNA can be stopped altogether.

There is yet another problem common to many analytical techniques that rely on binding of an analyte. The probability of binding is determined by the dissociation constant, Kd. For a highly selective binding agent, Kd might be as small as 10⁻⁹ M. However, many metabolites and proteins are present in living cells at much smaller concentrations than this. This is not a problem in DNA sequencing where the polymerase chain reaction can used to increase the concentration of an analyte, but there is no equivalent way of increasing the concentration of other analytes (e.g., proteins, amino acid metabolites). There is therefore a need for a device that concentrates and traps analytes to raise their effective concentration at the detector.⁶

In addition, it would also be desirable to develop a translocation control scheme that can be used with any charged polymer, that is simple to implement, and is compatible with schemes for the readout of chemical composition. These and other advantages are obtained in the translocation control scheme of the present invention.

### SUMMARY OF SOME OF THE EMBODIMENTS

Accordingly, to address at least some of the difficulties noted above, the present disclosure presents the following summary with respect to at least some of the embodiments disclosed herein.

In some embodiments, a device for controlling the transit of a molecule across a nanopore is provided and includes a first compartment, a second compartment, a first pair of electrodes comprising a first electrode provided in the first compartment and a second electrode providing in the second compartment, a partition separating the first compartment from the second compartment, an orifice provided in the partition, a second pair of electrodes arranged proximate the orifice, the second pair of electrodes being functionalized with molecules, and a tunnel gap comprising the spacing between the second pair of electrodes.

In some such embodiments, a voltage bias may be applied between the second pair of electrodes and may be configured to generate an electro-osmotic flow in a first direction for molecular transport.

In some such embodiments, an AC voltage of at least 1 kHz in frequency may be applied between the second pair of electrodes. Furthermore, the presence of a molecule in the tunnel gap may be detected by means of non-linear processing of the AC current signal.

In some such embodiments, a voltage bias may be applied between at least one of the first electrode and the second pair of electrodes and the second electrode and the second pair of electrodes, where the voltage bias is controlled by a circuit fed by a signal generated by the second electrode pair.

In some such embodiments, the voltage bias applied includes both an AC and a DC component.

In some embodiments, a device for controlling the collection and/or detection of molecules, is provided and includes a first compartment, a second compartment, a first pair of electrodes comprising a first electrode provided in the first compartment and a second electrode providing in the second compartment, a partition separating the first compartment from the second compartment, an orifice provided in the partition, and at least one orifice electrodes arranged proximate the orifice.

In some embodiments, a device for controlling concentration of analyte molecules in a nanopore is provided and includes a nanopore articulated with electrodes configured to generate an electro-osmotic flow of electrolyte in the pore by voltage biasing means, where the electro-osmotic flow is configured to at least one of capture and concentrate analyte molecules from a bulk reservoir provided on at least one side of the nanopore via consequent fluid flow from the bulk reservoir into the nanopore.

In some embodiments, a device for controlling the transit of a molecule across a nanopore is provided and includes a first compartment, a second compartment, a first pair of electrodes comprising a first electrode provided in the first compartment and a second electrode providing in the second compartment, a partition separating the first compartment from the second compartment, a nanopore provided in the partition, a second pair of electrodes arranged proximate the orifice, the second pair of electrodes being functionalized with molecules, and a tunnel gap comprising the spacing between the second pair of electrodes. In such embodiments, the first pair of electrodes may be biased to oppose a flow of molecules into the nanopore, and the second pair of electrodes may be biased to generate electro-osmotic flow into the nanopore.

In some embodiments, a nanopore device for controlling translocation of uncharged molecules is provided and includes a first compartment, a second compartment, a first pair of electrodes comprising a first electrode provided in the first compartment and a second electrode providing in the second compartment, a partition separating the first compartment from the second compartment, a nanopore provided in the partition, a second pair of electrodes arranged proximate the orifice, the second pair of electrodes being functionalized with molecules, and a tunnel gap comprising the spacing between the second pair of electrodes. In such embodiments, the second pair of electrodes may be biased so as to generate Stokes flow into the nanopore.

In some embodiments, a method for controlling the transit of a molecule across a nanopore is provided and includes providing a system or device according to one or another of the disclosed system/device embodiments, and applying a voltage bias between the second pair of electrodes configured to generate an electro-osmotic flow in a first direction for molecular transport. In some such embodiments, additional steps may include:
applying an AC voltage of at least IkHz in frequency between the second pair of electrodes;
detecting the presence of a molecule in the tunnel gap via non-linear processing of the AC current signal;
applying a voltage bias between at least one of the first electrode and the second pair of electrodes and the second electrode and the second pair of electrodes, where the voltage bias is controlled by a circuit fed by a signal generated by the second electrode pair,
the voltage bias comprises both an AC and a DC component.

In some embodiments, a method for controlling concentration of analyte molecules in a nanopore is provided and includes providing a system or device according to one and/or another of the disclosed system/device embodiments, and generating an electro-osmotic flow of electrolyte in the pore by voltage biasing means, where the electro-osmotic flow is configured to at least one of capture and concentrate analyte molecules from a bulk reservoir provided on at least one side of the nanopore via consequent fluid flow from the bulk reservoir into the nanopore.

In some embodiments, a method for controlling the transit of a molecule across a nanopore is provided and includes providing a system or device according to one and/or another of the disclosed system/device embodiments, biasing the first pair of electrodes to oppose a flow of molecules into the nanopore, and biasing the second pair of electrodes to generate electro-osmotic flow into the nanopore.

In some embodiments, a method for controlling translocation of uncharged molecules is provided and includes providing a system or device according to one and/or another of the disclosed system/device embodiments, and biasing the second pair of electrodes to generate Stokes flow into the nanopore.

These and some of the many other embodiments taught by the present disclosure will become even more evident with reference to the drawings included with the present application (a brief description which is provided below), and the detailed description which follows.

### BRIEF DESCRIPTION OF THE DRAWINGS

FIG. 1 is an illustration of translocation control using a molecular motor.

FIG. 2 is an illustration of translocation control with embedded electrodes in a solid state nanopore according to some embodiments of the present disclosure.

FIG. 3A illustrates an example of the trapping of a DNA base by recognition molecules tethered to electrodes spanning a nanopore.

FIG. 3B illustrates a distribution of translocation times (seconds) as a 63 base single-stranded DNA translocates a nanopore surrounded by a 10 nm thick Pd electrode functionalized with recognition molecules (bias=70 mV). The distribution time for the same nanopore and electrode without recognition molecules is shown by the black bars.

FIG. 4 illustrates a system for translocation control and readout scheme according to some embodiments of the present disclosure, in the form where tunneling electrodes are opposed to one another in the same plane.

FIG. 5 illustrates a planar tunneling junction configuration according to some embodiments of the present disclosure.

FIG. 6 illustrates a stacked tunnel junction configuration according to some embodiments of the present disclosure.

FIG. 7 illustrates a distribution of electric fields around a nanopore in a stacked tunnel junction configuration (a cross section of the device is shown, the full device is described by rotating this model around X-X) according to some embodiments of the disclosure, for the lower tunneling electrode at 0V and the upper at −0.5V (A), +0.5V (B) and 0V (C) with + and −0.1V applied to the upper and lower reference electrodes. This distribution is for the second tunneling electrode biased −0.5V with the other electrodes biased as shown. The field direction is shown by the arrows, and density of equipotential lines represents field strengths.

FIG. 8 illustrates contours of volume charge around a nanopore according to some embodiments (cross section of the device is shown, the full device is described by rotating this model around the vertical at 0 on the horizontal axis). This distribution is for the top tunneling electrode 47 biased 0.4V, the lower tunneling electrode 46 at 0V, the lower reference electrode at −0.05V and the upper reference electrode at +0.05V. Contours are shown only for positive charge—the “holes” near the central axis of the nanopore correspond to regions of accumulation of negative charge (though the average is everywhere positive).

FIG. 9A illustrates different domains of the nanopore (numbers on left) according to some embodiments.

FIG. 9B is a table (Table 1), listing the volume charge, electric field and force on each volume of fluid. Note the very large reversal of force in between the tunneling electrodes (region 5) where electro-osmotic flow overcomes the electrophoretic force.

FIG. 10 illustrates a particle velocity through the nanopore as a function to the voltage applied across the tunneling electrodes, V3, according to some embodiments.

FIG. 11 illustrates an embodiment of the present disclosure in which electro osmotic forces and electrophoretic forces oppose one another. The reference electrodes can be biased in a “wrong direction” but flow through the nanopores still occurs.

FIG. 12 illustrates measured count rates showing capture of DNA molecules as a function of bias for a Bare SiN nanopore (squares) the same pore with a bare Pd electrode surrounding it (filled circles), and the same pore with the electrode functionalized with the 4(5)-(2-mercaptoethyl)-1H imideazole-2-carboxamide reader molecules, according to some embodiments of the present disclosure.

FIG. 13 illustrates the capture scheme for concentrating molecules at the entrance to the nanopore, according to some-embodiments of the present disclosure.

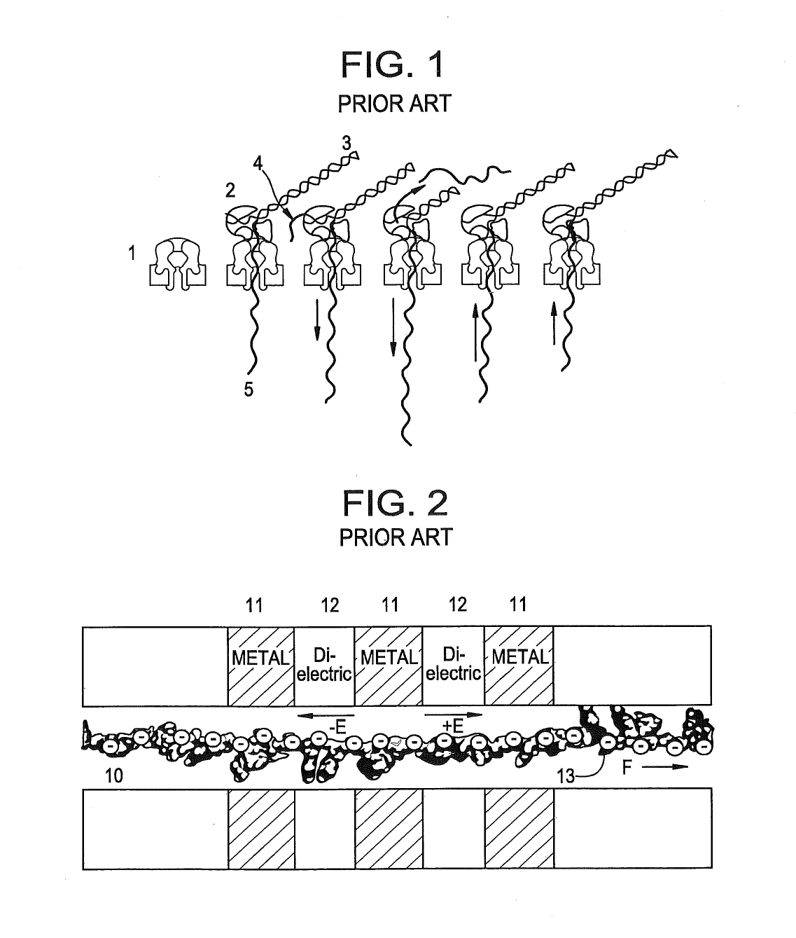

### DETAILED DESCRIPTION OF SOME OF THE EMBODIMENTS

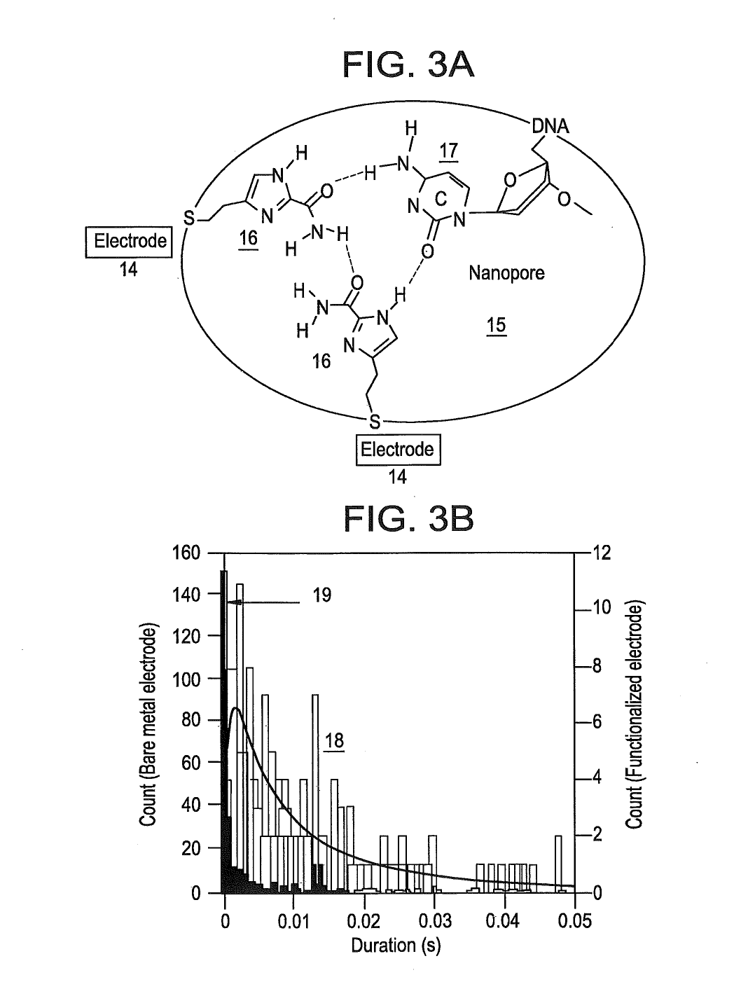

The basis of some of the embodiments of the current disclosure is the trapping of target molecules by a recognition reagent tethered to tunneling electrodes, known as recognition tunneling.⁷ In a series of earlier disclosures, W02009/117522A2, WO 2010/042514A1, W02009/117517, W02008/124706A2, and W02011/09141, each of which is incorporated herein by reference, a system was disclosed where nucleic acid bases could be read by using the electron tunneling current signals generated as the nucleobases pass through a tunnel gap functionalized with adaptor molecules. A demonstration of the ability of this system to read individual bases embedded in a polymer was given by Huang et al.⁸ In the paper by Huang et al. it was shown how dynamic force spectroscopy can measure the off rate of a target molecule trapped by a pair of recognition molecules. This trapping is illustrated in FIG. 3A using the specific example of a C base in DNA 17. The recognition molecules, 4(5)-(2-mercaptoethyl)-1H imideazole-2-carboxamide, 16 are covalently tethered to electrodes 14 that are separated by 2 to 3 nm. The recognition molecules 16 form a hydrogen-bonded complex with the base 17 as the DNA passes through a nanopore 15 spanning the space between the electrodes.⁹

Recognition tunneling not only enables a read of the sequence, but in some cases, can also trap the analyte molecule in the tunnel gap. Even with carefully selected temperatures, solution viscosities and biases, the slowest translocation times (in a ˜4 nm diameter pore) that have been achieved in a solid state nanopore are about 0.3 μs per base (for double stranded DNA)¹⁰, much too fast for the sequence to be read electronically.

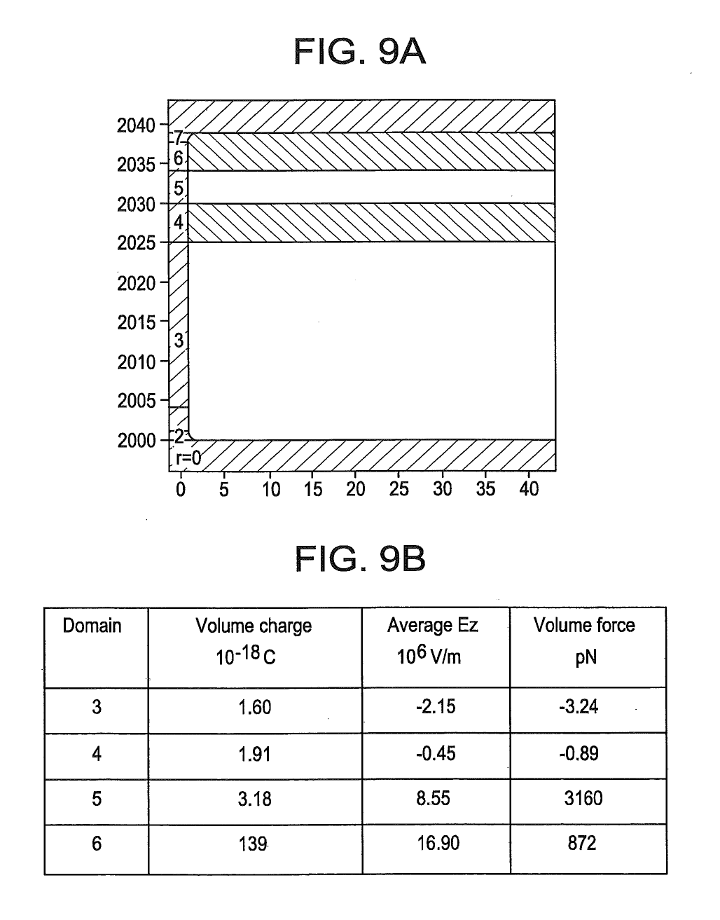

An exemplary illustration for how translocation is slowed in a functionalized nanopore, the binding kinetics of the analyte to the recognition molecules are considered. The off-rate of the bonded complex (FIG. 9) k_{off}, depends upon an applied force according to

$${k}_{off} = {k}_{off}^{0} exp ( \frac{{Fx}_{TS}}{{k}_{B} T} ) \qquad ( 1 )$$

where k_{off}⁰ is the off rate at zero force, F is the applied force breaking the trapping bonds, x_{TS} is a parameter that describes the barrier to bond breaking (the distance to the transition state for bond breaking), k_{B} is Boltzmann's constant and T is the absolute temperature. Direct measurements of k_{off}⁰ in a nanometer-scale gap (using dynamic force spectroscopy⁸) shows that it is about 0.3 s⁻¹. Thus, with no external force applied, a DNA base will remain in a recognition tunneling gap for about 3 seconds. This period of time is a result of the confinement of the complex.^{11,12}

The same set of dynamic force spectroscopy measurements⁸ yields x_{TS}=0.78 nm. Since the force on a DNA molecule generally depends on the voltage applied across a nanopore according¹³ to F=0.24 pN/mV, the off rate at any externally applied bias can be calculated. For example, if a dwell time of 10 ms per base is desirable to read sequence accurately, then k_{off} of should be about 100 s⁻¹ or a 333-fold increase over k_{off}⁰, so that

$$\frac{{Fx}_{TS}}{{k}_{B} T} = ln ( 333 ) = 5.8 .$$

At 300K, k_{B}T=4.2 pN·nm, so Fx_{TS} should be about 24.4 pN·nm. With x_{TS}=0.78 nm, F=31.2 pN, a force given by a voltage of 130 mV. Accordingly, the translocation is slowed to residence times of 10 ms per base by chemical binding compared to 0.3 μs per base in an unfunctionalized pore at a similar bias,¹⁰ a factor of 30,000 time slower.

Experimental data supports a finding that functionalization of the electrodes slows translocation compared to translocation past bare metal electrodes (FIG. 3B). In addition, translocation times scale linearly, not exponentially with applied voltage, indicating that friction dominates over activated binding and unbinding. A distribution of the current blockade times taken with a nanopore surrounded by a Pd electrode functionalized with 4(5)-(2-mercaptoethyl)-1H imideazole-2-carboxamide is shown by the unfilled bars 18 in FIG. 3B. The data is for a 63 nt single stranded DNA with an applied bias of 70 mV. The fit (solid line) is to a log normal distribution with a peak at 10±1 ms, corresponding to about 0.2 ms dwell time per base (though many events are longer than this).

In contrast, the median value of blockade time for unfunctionalized electrodes (black bars 19 in FIG. 3B) is about 0.5 ms for the same pore with the same DNA also at 70 mV bias. This corresponds to about 8 μs per base. This is still >20 times slower than the slowest times reported for a non-metalized pore (and double stranded DNA) which is believed due to binding of single stranded DNA to the metal electrode.¹⁴ Thus, with metal electrodes functionalized with 4(5)-(2-mercaptoethyl)-1H imideazole-2-carboxamide and placed in close proximity to a nanopore, DNA translocation may be slowed by a factor of about 1000 times compared to translocation through a solid state nanopore with no metal electrode and no functionalization. The recognition tunneling geometry serves to slow translocation adequately for sequence reads, provided that a limited bias (e.g., 10-100 mV range) can be applied across the pore. However, the need to apply a bias across tunneling electrodes in some embodiments may complicate the application of an arbitrary translocation bias across the nanopore. This can be addressed as noted below.

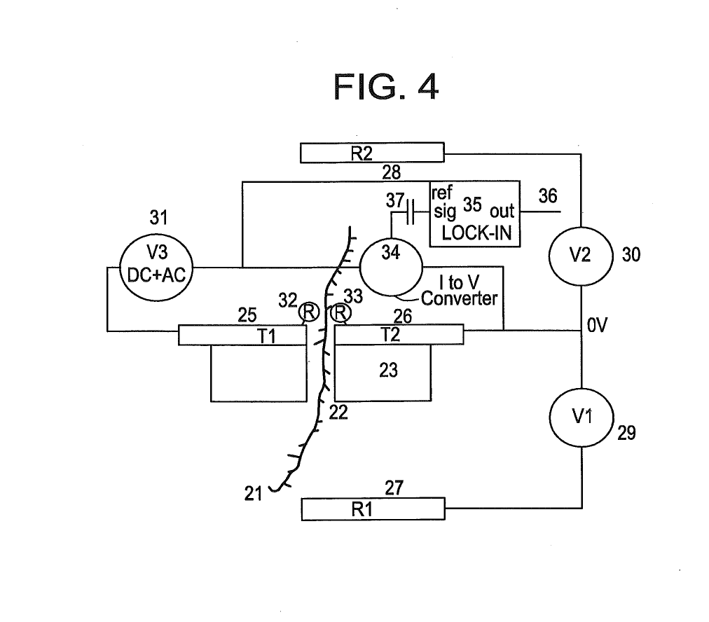

In some embodiments, generation of signals by recognition tunneling includes a voltage across the tunnel gap of between about 0.1 to about 0.5V,¹⁵ which is greater than the translocation bias values disclosed above. Referring to FIG. 4, in some embodiments, the overall translocation bias is applied across a pair of reference electrodes R1, 27 and R2, 28 immersed in electrolyte solution on each side of the pore. The total bias applied between the two reference electrodes is V1+V2 (V1, 29, V2, 30 on the figure). Since the nanopore itself contains tunneling electrodes (T1, 25, and T2, 26), in some embodiments, the potential of these electrodes is defined with respect to the reference electrodes (in some embodiments, if this is not done, then ions and charged molecules can adsorb onto the metal surface in such a way as to alter its potential so as to oppose translocation). In some embodiments, upon the two tunneling electrodes being at the same potential (V3, 31, =0) then, ideally V1 is set about equal to the magnitude of V2 (where V2<0) so that the potential of the nanopore electrodes 25 and 26 lies midway between the two reference electrodes 27 and 28.

According to some embodiments, if the molecules to be translocated are placed in the lower reservoir, then, in the case of DNA (negatively charged) making V1 more negative results in a faster capture of molecules, whereas increasing V2 results in more rapid pulling of the molecules out of the junction formed by T1 25 and T2 26. (together with the attached recognition molecules, R, 32, 33). However, if a bias is applied across the tunnel junction that is greater than the magnitude of V1+V2, then one tunneling electrode or the other is higher (or lower) in potential than either R1 or R2, since typical tunneling voltages are greater (500 mV) than translocation potentials (e.g., magnitude of V1+V2=50 mV). If either T1 or T2 are below R1 in potential, then DNA molecules are repelled from the nanopore (if electrostatic forces alone are considered). If either T1 or T2 is above R2, then DNA molecules will not be pulled away from the gap rapidly, once again, in the limit that electrostatic forces alone are considered.

In some embodiments, one solution is to operate the tunnel gap with an alternating-current (AC) bias. Specifically, if the frequency of the AC bias is above the dielectric response frequency of DNA (typically a few kHz¹⁶⁻¹⁹) then the effect of an AC bias V3 31 on translocation may be small. As shown below, some embodiments provide for V3 as a combination of a DC voltage with an AC signal imposed. In some such embodiments, the tunnel current is detected with a peak amplitude detector or lock in, as is well known in the art. This is because the time average of the current signal is zero with an AC bias applied. Ref. no. 34 is a current-to-voltage converter (trans-impedance amplifier), according to some embodiments, that generates a voltage signal proportional to the tunnel current flowing across the junction between T1 and T2. In some embodiments, the response of this converter is 1V out for 1 nA of current flowing through the device. Accordingly, the AC voltage out is fed to the signal input of a lock-in detector, 35, the DC component being blocked by a capacitor, 37. The AC driving voltage is used as a reference signal for the lock-in. Exemplary values of this bias are in the range of about 100 mV to about 1000 mV, peak to peak, with 500 mV peak to peak preferred. Exemplary frequencies may be in the range of about 1 kHz to about 100 kHz with about 20 kHz being a preferred frequency according to some embodiments. Signal averaging times for the resultant DC signals according to some embodiments are in the range of between about 5 ms to 50 about microseconds, with about 500 microseconds preferred according to some embodiments. These times may be set in the lock-in 35 to generate an output voltage 36, thereby permitting (in some embodiments) averaging over a few cycles of the AC modulation signal while retaining dynamic features of the tunneling signal that are essential to allow identification of the chemical species in the gap.²⁰ It will be recognized to those of ordinary skill in the art, that the lock-in may be replaced with a simple peak detection circuit (diode and capacitor) with a resistor used to set the signal averaging time constant.

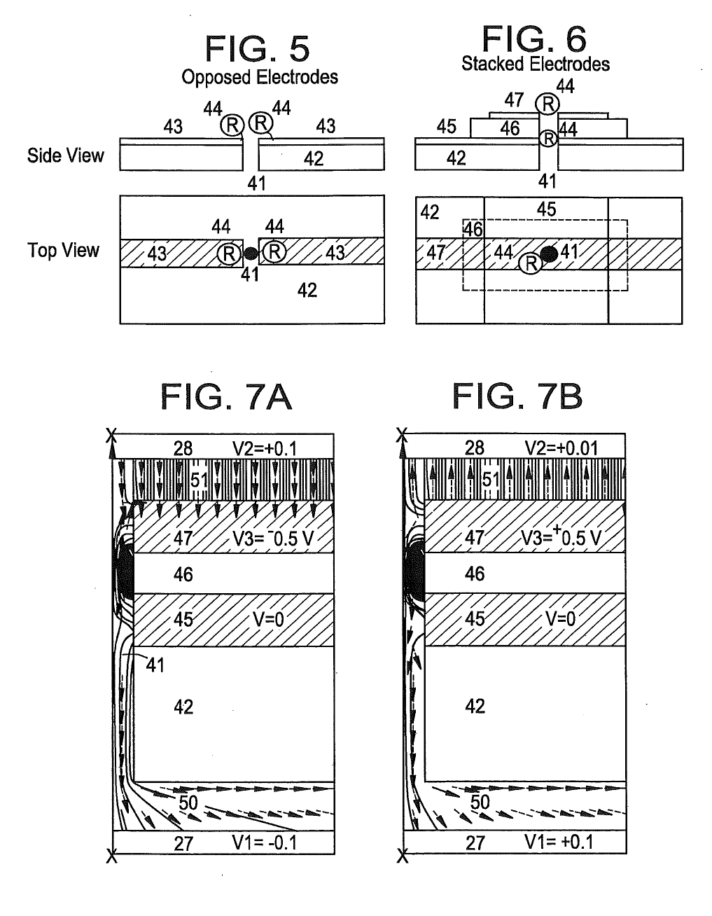

FIGS. 5 and 6 illustrate two exemplary arrangements for the tunnel junctions in the nanopore according to embodiments of the disclosure. FIG. 5 shows a planar configuration according to some embodiments, in which a wire, of about 10 to about 100 nm in width, is cut to form a pair of electrodes 43 that span a nanopore 41 in a membrane 42. In some embodiments, the membrane is typically between about 10 to about 100 nm thick and made of silicon nitride or an oxide of silicon. The gap between the electrodes is between about 2 nm and about 3 nm and the nanopore may include a similar diameter. FIG. 6 shows a planar ‘stacked’ configuration according to some embodiments, previously described in U.S. application No. 61/711,981 (herein incorporated by reference). In FIG. 6, the two electrodes 45 and 47 are separated by a dielectric layer 46 of about 2 nm to about 3 nm in thickness, with Al₂O₃ being the preferred material, deposited by atomic layer deposition (according to some embodiments). The electrodes (45 and 47) may be about 4 nm to about 10 nm thick Pd metal deposited on a thin (0.5 nm) Ti adhesion layer. The sandwich sits on a SiN substrate 42, typically about 10 to about 100 nm in thickness. A nanopore 41 (or other gap) is drilled through the entire assembly to expose edges of the electrodes 45 and 47. In some embodiments, the electrodes may be functionalized by immersing the entire device overnight in an ethanol solution of the recognition molecules 44. This planar configuration includes unexpected properties, leading to a solution of the problem of using a large tunnel bias, and giving the ability to trap even neutral molecules in the gap by collecting them from a large range of distances.

The motion of DNA or any other charged polymer in the nanopore is complex since the actual force on the DNA has contributions besides the electrophoretic attraction of the DNA to the positively polarized electrode. Specifically, the forces on the DNA can include:
(a) The drag force, which depends on the size and speed of the molecule.
(b) The electrophoretic force, proportional to the DNA charge, which, to first approximation, depends only on the voltage along the pore (the DNA charge increases with length, but the field in the pore, for a given voltage, decreases with pore length).
(c) The dielectrophoretic force, which depends on the gradient of the magnitude of an AC field, its frequency, its size and dielectric and conductive properties of the DNA relative to the electrolytic environment.
(d) Random thermal forces.

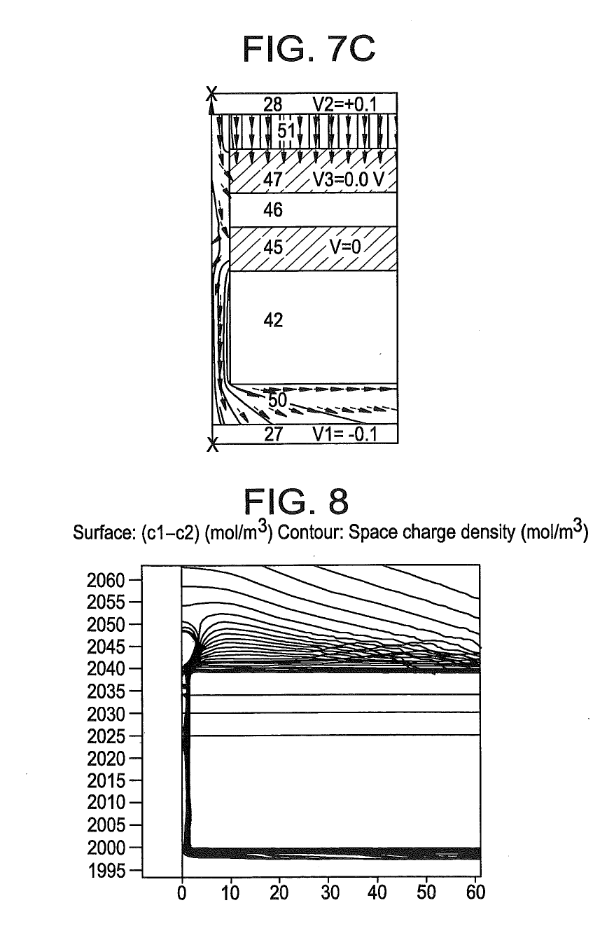

All of these contributions have been considered in a finite element analysis of DNA translocation in the stacked electrode device, according to some embodiments, shown in FIGS. 7-9. In FIG. 7, half of the vertical cross section of the pore geometry is shown due to the cylindrical azimuthal symmetry of the configuration. The three dimensional structure is generated by rotating the model around the axis line marked X-X. Half the nanopore 41 is shown on the left of FIGS. 7A-C and 8. The silicon nitride substrate is 42. The lower Pd electrode is here assumed at V=0, is 45. The dielectric Al₂O₃ layer is 46. The top Pd electrode is 47. A salt solution in the lower chamber 50 is in contact with a lower reference electrode 27. Similarly, a salt solution in the upper chamber 51 is in contact with an upper reference electrode 28. The electric fields in 1M KCl are shown by arrows on the figures. The series (FIG. 7A-C) show how the field distribution changes as the top electrode 47 is biased at −0.5V, 0V and +0.5V with the top reference electrode biased at +0.1V and the bottom reference electrode biased at −0.1V. DNA of 60 bases or longer is, according to some embodiments, translocated through the pore, even when a strong electric field opposing electrophoresis is between the electrodes 47 and 45 (i.e., for the case where V3=−0.5V). This is because, while the electric field in this region acts to push DNA out, the electro-osmotic flow (just in this region) acts in the opposite direction.

Accordingly, the DNA is drawn into the pore by the attractive electrophoretic force in the vicinity of the electrode 45, then becomes trapped (or in some embodiments, even pushed back down again) by the barrier presented by the reversed field between electrodes 47 and 45. As soon as a fluctuation drives it into next region of electric field reversal (above electrode 47), it can then be swept up by the top electrode 28. In some embodiments, a similar pattern occurs with smaller V3 though now the DNA is trapped in the region of reversed field between electrodes 45 and 47 for less time. The behavior can be different when V3=0V (FIG. 7C), where now no electric field is present in the region between the two electrodes 45 and 47. The result is that the DNA is often trapped, sometimes being ejected from the pore by the electrosmotic flow back into the lower reservoir 50. In some embodiments, this effect is size-dependent.

In some embodiments, in the reverse situation, where V3>0 (FIG. 11) translocation still occurs. In this case, there is a strong electric field aiding translocation in the gap between electrodes 45 and 47. However, while the DNA can stick to the most positive electrode 47, it may diffuse out of the nanopore quickly, diffusing out over the top surface of the electrode 47.

The physical mechanism leading to this counterintuitive behavior is discussed in details in FIGS. 8-10. The metal electrodes at the top of the SiN nanopore (which has a negatively charged surface facing the solvent) and the surface of the upper Pd electrode, 47, at negative potential, also gets strongly negatively charged, and both these surface charges induce positive volume charge in the nanopore. The distribution of the volume charge is not uniform (FIG. 8), with even appearance of the regions of negative volume charge around the axis (white regions in the pore). The volume force acting to these charges is proportional also to the electric field and for positive charges directed like the field. Since the fields changes its direction (FIG. 7A) in the pore, there are two opposite forces acting to the solvent. Due to the water non-compressibility and continuity, the whole liquid will move in the direction of the stronger force. The pore is divided into several domains (FIG. 9) and show in Table I the total volume charge, average longitudinal component of the electric field and total volume force for each domain, in case of V3=−0.4V (with V2-V1=100 mV). The domain volume forces pointing up, i.e. from negative to positive electrode prevails, causing the flow of the solvent in that direction.

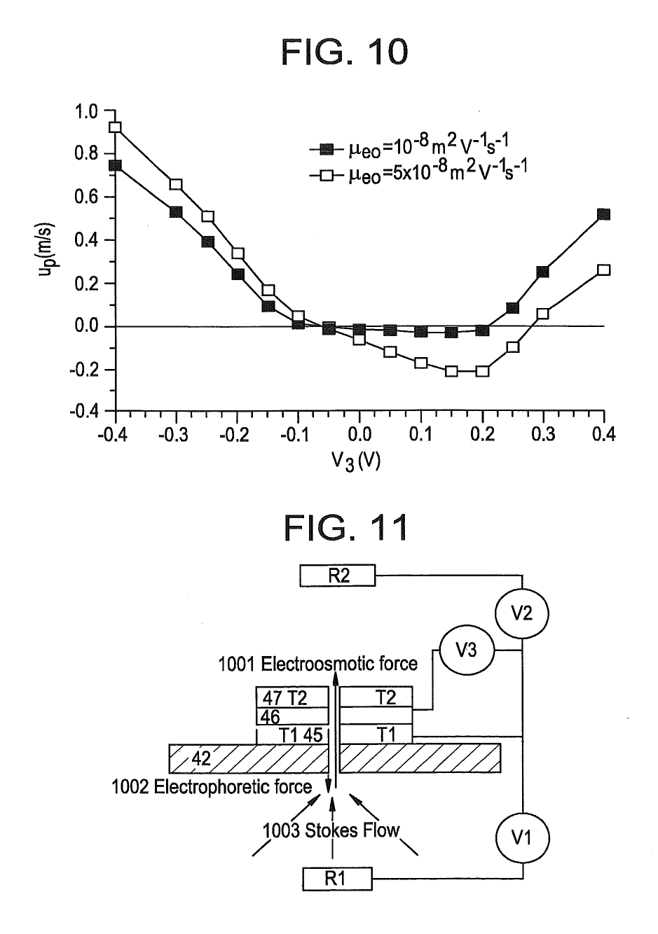

FIG. 10 shows the translocation speed as functions of the bias across the tunneling electrodes, V3, for the “stacked” tunnel junction geometry shown in FIG. 6, according to some embodiments. Since this arrangement is to a single particle model of DNA, it does not represent the additional friction that results from the extended chain or the forces on the parts of the chain outside the nanopore. It also does not include the frictional forces that result from recognition molecules (32, 33 in FIG. 4) binding to the DNA.

The translocation velocity of a DNA in water through a nanopore reacts to the changes in the electric field and mobility, at the scale of ps. The instantaneous velocity ν̅ can be therefore expressed in terms of the instantaneous values of the electric field Ē, electrophoretic mobility μₑₚ and electro-osmotic velocity μ̅ in the form

v̅=ū+ v̅ₑₚ=ū+μₑₚ E̅

To estimate the order of magnitude of the translocation time is approximated

μₑₚ≅3·10⁻⁴ m²/sV

See Earle Stellwagen, Yongjun Lu, and Nancy C. Stellwagen, Nucleic Acids Res. 2005; 33(14): 4425-4432; Nancy C. Stellwagen and Earle Stellwagen, J Chromatogr A. 2009 Mar. 6; 1216(10): 1917-1929., Phys Rev E Stat Nonlin Soft Matter Phys. 2008 August; 78(2 Pt 1): 021912.

The μₑₚ depends weakly on the viscosity of the electrolyte, electrolyte concentration and effective (screened) charge of the DNA, the DNA length, the length of the nanopore and its radius, but for the purposes of illustration, it is assumed constant.

Referring to Table 1 with FIG. 9 and FIG. 10, with z-axis vertical from the bottom up, the z-component of electrophoretic velocity νₑₚ in various domains of the pore in FIG. 9 (when V3=−0.4V) takes values from 6.45 cm/s to −0.5 m/s, while the average z-component of electro-osmotic velocity u has the same value of μₛ=0.75 m/s for all domains due to the water incompressibility. This leads to the estimates of the average translocation times through various domains (3,4,5,6) of ˜31 ns, 6.8 ns, 8 ns and 20 ns for a base, giving the total of T>66 ns. In cases in FIG. 10 when u=0 (when V3=−0.1V or +0.22V), only electrophoretic velocity determines the translocation time which is determined for this case at T>1′/base. By choosing values of V3 between −0.1V and 0.22 V, the electro-osmotic velocity is negative, opposing the electrophoresis and the translocation time can be made almost arbitrarily long.

Since, because electroosmotic Stokes flow law, flow of liquid into the nanopore brings molecules into the pore from a large range of distances, owing to the incompressibility of the liquid, while the electrophoretic force only acts near the pore, the reference electrodes R1 and R2 can be biased the “wrong way” and molecules can still be captured and translocated. This is illustrated in the exemplary embodiments shown in FIG. 11, where the stacked tunnel junction configuration is used, and the electrodes are labeled as in FIG. 6, with the voltages V1, V2 and V3 defined as in FIG. 4. For example, in the case where negatively charged DNA molecules are placed in the lower reservoir between R1 and the partition 42, and V1 is arranged so that R1 is positive with respect to T1 45, the electrophoretic force 1002 acts to drive the DNA molecules away from the nanopore. However, the electro-osmotic force that results when T2 47 is made negative with respect to T1 45 now pulls molecule into the pore, provided that the electro-osmotic force exceeds the electrophoretic force. Thus, in these conditions the translocation can be made substantially arbitrarily slow. Furthermore, by applying a large V1 in the “wrong” direction, a substantial V3 can be applied to generate large tunneling signals. Because of the long range of the Stokes flow 1003 molecule can still be captured efficiently because the electrophoretic force opposing entry to the nanopore only acts in the immediate vicinity of the nanopore.

These behaviors are replicated in some embodiments when the applied bias V3 is an AC sine-wave. The DNA translocates according to the value of V3 at the time when it enters the pore. This is the case in such embodiments since translocation times are much shorter than a period of the AC waveform in these simulations.

Accordingly, in some embodiments, with the tunneling detection carried out with an AC bias of greater than a threshold frequency (in some embodiments, greater than about 10 kHz), DNA translocates when the bias reaches an optimal value. To estimate the frequency required for some embodiments, more realistic measured translocation times are considered in a functionalized tunnel junction (FIG. 3B). In some embodiments, in order to read a sequence, many cycles per base are needed. Accordingly, the peak of the distribution shown in FIG. 3B corresponds to 0.16 ms/base. To sample each base 10 times, according to some embodiments, requires an AC frequency of about 63 kHz.

In some of the embodiments discussed above, with just electrophoretic forces and no recognition molecules, the translocation time is microseconds for a 60 base DNA because the chemical drag imposed by recognition molecules was not included in the model. For example, to simulate the effect of an AC voltage for V3 where the frequency corresponds to many cycles during the time each base spends in the gap, the frequency of the AC signal is set to about 100 MHz. This results in a signal which does not change the translocation probability as controlled by the DC voltages alone, V1, V2 and the DC component of V3. Therefore, according to some embodiments, a small DC value of V3 can be used to control the translocation rate, while a much larger superimposed AC voltage generates the required magnitude of tunneling signal for readout of the sequence.

According to some embodiments, V1 and V2 can be controlled externally by a computer program fed the tunneling signal as the input used to control the translocation voltage values, enabling active control of these potentials. While active control of translocation potential has been proposed before (see Keyser²¹ and references therein), such proposals were only in the context of measuring ion current blockade as the signal used to control the potential applied across the nanopore. To that end, the ability to measure tunneling signals by the means described herein according to some embodiments opens a new avenue for translocation control. For example, according to some embodiments, V1 may be made greater (0.1-0.5 volts) until a tunneling event is signaled by the detector output 36. At that point, V1 may then be reduced to prevent further capture while V2 may then be adjusted to give the desired rate of translocation.

In some embodiments, V3 31 (in FIG. 4) may be set to a voltage of between about −0.1V and about −0.5V. V1 29 may be set to a value between about −0.01 and about −0.1V and V2 30 may be set to a value of between about +0.01 and about +0.1V. Accordingly, when a tunneling signal indicates that a molecule is present in the gap, V2 may be dropped to −0.5V, matching the bias applied to the electrode 47 and stalling the molecule in the gap until a recognition tunneling signal is recorded from the first trapped base. V2 may then be briefly returned to a value between about +0.01 and about +0.1V and then dropped again to allow reading of the next base in the sequence, and so on.

In some embodiments, V3 may be an AC voltage of about >10 kHz in frequency and between about 0.1 and about 1V in peak to peak amplitude. V1 29 may be set to a value between about −0.01 and about −0.1V and V2 30 may be set to a value between about +0.01 and about +0.1V. Once a capture event is detected by a tunneling signal, V1 may then be reduced to prevent further capture and V2 may be reduced to obtain a desired translocation rate.

In some embodiments, the sign of V1 can be changed altogether once a molecule is captured, so that both R1 and R2 operate to pull on the ends of the molecule. Accordingly, with equal and opposite forces pulling on the molecule, the molecule may be stopped in the pore altogether. The advantage in such embodiments is that a large (stretching) force may be placed on the molecule, reducing thermal fluctuations substantially, so that even a bias difference substantially less that kT (i.e., much less than 25 mV for V1-V2 where V1 and V2 act in opposite directions) may be used to translocate the molecule, while suppressing thermal fluctuations in the position of the molecule because the potential differences across the front and back entries to the nanopore will be much larger than thermal fluctuations in energy. Thus, the range of potentials over which translocation may be actively controlled is greatly enhanced.

In some embodiments, the reference electrodes are biased so as to oppose transport into the nanopore, but the tunnel bias is configured to generate an electro-osmotic flow that can overcome this opposing force and drag molecules into the pore, but at a much slower speed because of the opposing force. The electro-osmotic flow results in efficient capture of molecules because of the much longer range of Stokes flow compared to the short range of the local electric fields in the salt solution.

While the present disclosure illustrates systems and operation thereof with DNA molecules, it will be recognized that it will work with any charged or neutral polymer carrying a net positive or negative charge or no charge at all. In particular, in a system dominated by Stokes flow as just described above, neutral molecules will be concentrated and dragged into the nanopore

Accordingly, as is evident from FIG. 7, there is an electric field (which may be relatively substantial, significant) that may extend several pore diameters from the entrance and exit of the pore. A particle entering this region may be swept into the nanopore where it will translocate, or be trapped, depending on the values of V2 and V3. In some embodiments, once the analyte is in the tunnel junction, binding by the recognition reagents is highly probable, because the effective concentration of binding partners is very high. To illustrate this, consider a tunnel junction of volume (2 nm)³ which is 8×10⁻²⁷ m³ or 8×10²⁴ liters. One molecule in this volume corresponds to about 10²³ molecules per liter or ˜0.1M. The diffusion limited value for Kₒₙ is 10⁹ M⁻¹s⁻¹ with a lower experimentally determined limit in systems where binding is difficult being 10⁶ M⁻¹s⁻¹. Thus, even at this lower limit, the binding is very rapid (1/[KₒₙC], where C is the concentration, giving a time of 10 μs) consistent with experimentally determined limits.²⁰ Thus, every molecule that reaches the region of the specific electric field near the pore may be detected. Far from the pore, the field is appreciably small.

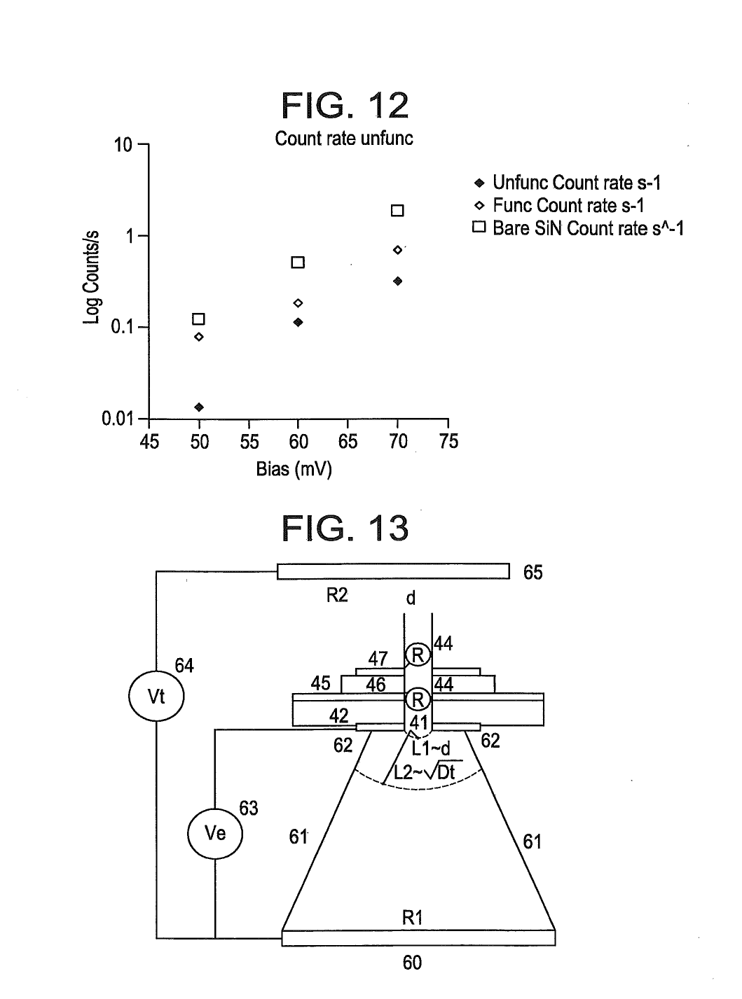

To illustrate this, consider the field in the pore, which is on the order of about 0.1V/10 nm or 10⁷ V/m. Spaced from the pore, in some embodiments, one or two pore diameters, 10 nm), the current density (and hence field) will drop in the ratio (r/R)² where r is the radius of the nanopore and R is the radius of the channel leading to the pore. Accordingly, if R™1 μm and r˜1 nm, then the field in the reservoir spaced away from the pore (e.g., one or two pore diameters, 10 nm) is less than about a volt per meter. The drift velocity of a small molecule (with mobility 10⁻⁸ m²/Vs) may therefore be about 10 nm/second. Therefore, in the absence of Stokes flow, the motion of molecules in the reservoir far from the pore is dominated by diffusion because the electrophoretic drift velocity is sufficiently small at these values of current determined by the nanopore geometry. The distance a molecule diffuses, L2, in a time t is given by L2=\sqrt{Dt} where D is the diffusion constant. For a small molecule, D˜10⁻¹⁰ m²/s. Thus, molecules with velocity vector components directed towards the pore will be trapped over a volume ˜(L2).³ In such embodiments, this can limit the capture rate. FIG. 12 shows measured capture rates, according to some embodiments, for a bare SiN pore, a pore with a Pd electrode incorporated and a pore with a Pd electrode that has been functionalized with recognition molecules. At small values of bias between R1 and R2 (V1+V2) the count rates are relatively small (in some embodiments, a few counts per minute) even at the 100 nM concentrations (for example).

The small capture volume for electrophoresis is illustrated in FIG. 12. In this figure, L1 represents the radius of a hemisphere in which the electric field near the pore is large (on the order of 10⁶ V/m). Molecules that diffuse into this hemisphere will pass into the nanopore. A fraction of the population, f, will have velocity vectors pointing towards this high field region where f<0.5, with a likely value of ˜0.1 depending on the details of the geometry of the reservoir and pore. Thus, the number of molecules, N, caught in the nanopore in t seconds is approximately

N˜fC(Dt)^{3/2}

where C is the concentration of the target analyte. Having N≧1 leads to

$$C \geq \frac{1}{{f ( Dt )}^{\frac{3}{2}}}$$

molecules/m³ or

$$C \geq \frac{{1.610}^{- 27}}{{f ( Dt )}^{\frac{3}{2}}}$$

in units of moles/liter. For t=1 s, D=10⁻¹⁰ m²/s and f=0.1, C≧16 pM. Thus, even in acquisition times of just one second, the lower concentration limit of some embodiments of the present disclosure is an improvement over many antibody-based detection systems⁶ (and antibody-based systems require a priori knowledge of the analyte). This lower limit, Cₘᵢₙ; scale as t^{3/2}, so increasing acquisition time to 100 s lowers Cₘᵢₙ to 16 fM.

In some embodiments, this lower limit may be further lowered (in a given time) upon the electric field in the reservoir being increased beyond the small field generated by the current through the nanopore. Accordingly, this may be accomplished with an additional electrode 62 being placed on the lower surface of the nanopore, restricted (in some embodiments) to an area close to (e.g., within a few microns) the nanopore. by applying a large bias (0.05 V or larger) between a lower reference electrode R1 60 and the electrode 62 with a bias Ve 63, charged molecules can be driven to accumulate on the electrode 62. The reservoir walls 61 are optimally shaped to void dead spots where the field generated by Ve is smaller owing to geometry. Once molecules have been concentrated on the lower surface electrode 62, the bias of the lower reference electrode 60 and the upper reference electrode 65 can be returned to values optimal for translocation.

In some embodiments, when electro-osmotic flow dominates the transport (over diffusion; see conditions in FIG. 9B—Table 1), the following considerations apply. For example, the electric potential at the cis side of the pore of radius R and length L is²²

$$V ( γ ) = \frac{{R}^{2}}{2 L γ} δ V$$

where δV is the bias voltage across the pore. The “radial” electric filed is

$${E}^{{r}^{i}} ( i ) = - {∇}^{r} V ( r ) \sim \frac{{R}^{2}}{{r}^{2 }} \frac{δ V}{2 L}$$

and the electrophoretic velocity of DNA is

$${u}_{E} ( r ) E \text{:} {μ}_{E} \frac{{R}^{2}}{{r}^{2}} \frac{δ V}{2 L} .$$

The electrophoresis dominates the diffusion in the capture to the pore when

$${u}_{E} ( r ) > D / 2 r ,$$

where D/2r is the average one-dimensional diffusion velocity in direction of r. The critical radius is thus

$${r}_{E} = \frac{{μ}_{E}}{D} \cdot {R}^{2} \frac{δ V}{L} = \frac{\text{?}}{D} {R}^{2} {E}_{o} , \\ \text{?} \text{indicates text missing or illegible when filed}$$

where Eₚ is the average electric field inside the pore.

Alternatively, in some embodiments, upon

$$\text{?} = \text{?} {E}_{p}$$

$$\text{?} \text{indicates text missing or illegible when filed}$$

with the electro-osmotic mobility in the pore, the continuity and incompressibility of the solvent requires that the solvent flow at the cis side at a distance r according to some embodiments is approximately

$$\text{?} ( r ) = \frac{{R}^{2}}{2 {r}^{2}} \text{?} = \text{?} \frac{{R}^{2}}{2 {r}^{2 }} {E}_{p} . \\ \text{?} \text{indicates text missing or illegible when filed}$$

This assumption may be confirmed (approximately) by numerical calculations. The water flow can dominate the diffusion in the capture if

$$\text{?} ( r ) > D / 2 r$$

$$\text{?} \text{indicates text missing or illegible when filed}$$

which yields the electro-osmotic critical radius

$$\text{?} = \frac{\text{?}}{D} {R}^{2} {E}_{p} . \\ \text{?} \text{indicates text missing or illegible when filed}$$

Finally, the electro-osmotic capture in some embodiments can dominate the electrophoretic one if r_{EO}>r_{g}, i.e., if electrophoretic mobility of the DNA in the pore is bigger than the electrophoretic one,

$$\text{?} > {μ}_{E} . \\ \text{?} \text{indicates text missing or illegible when filed}$$

The velocity of the DNA translocating through the pore can be

$$( \text{?} + \text{?} ) {E}_{p} , \\ \text{?} \text{indicates text missing or illegible when filed}$$

where the mobilities contain the sign defining the direction of the electric field and direction of the solvent electro-osmotic flow in various domains, as shown in FIG. 9.

Finally, the capture rate in case of electrophoresis and electro-osmosis, i.e., the flux of particles through the pore orifice, in some embodiments is

$${N}_{E , EO} - C π {R}^{2} μ {E}_{p} = {Cr}_{E , EO} π D$$

respectively, which corresponds to the Smolochowsky formula for the diffusion flux at the target of radius r_{E.EO}. Again assuming N1 leads to

$$C \geq \frac{1.6 \cdot {10}^{- \text{?}}}{{r}_{E \cdot EO} π D} = \frac{1.6 \cdot {10}^{- \text{?}}}{π {R}^{2} μ δ V} L$$

$$\text{?} \text{indicates text missing or illegible when filed}$$

which in some embodiments yields

C≧50 pM

where mobility was assumed 10⁻⁸ SI units. This condition is the same order of magnitude as the one following from the diffusion assumption, but the capture may work for neutral molecules. Extension of the time to 100 s reduces this limit to close to fM concentrations. It is noted again that because the electro-osmotic capture mechanism in some embodiments is by means of fluid flow and not electrophoresis, neutral molecules can be captured.

Various implementations of the embodiments disclosed, in particular at least some of the processes discussed, may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

Such computer programs (also known as programs software, software applications or code) include machine instructions for a programmable processor, for example, and may be implemented in a high-level procedural and/or object-oriented programming language, and/or in assembly/machine language. As used herein, the term “machine-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the subject matter described herein may be implemented on a computer having a display device (e.g., a CRT (cathode ray tube) or LCD (liquid crystal display) monitor and the like) for displaying information to the user and a keyboard and/or a pointing device (e.g., a mouse or a trackball) by which the user may provide input to the computer. For example, this program can be stored, executed and operated by the dispensing unit, remote control, PC, laptop, smart-phone, media player or personal data assistant (“PDA”). Other kinds of devices may be used to provide for interaction with a user as well; for example, feedback provided to the user may be any form of sensory feedback (e.g., visual feedback, auditory feedback, or tactile feedback); and input from the user may be received in any form, including acoustic, speech, or tactile input.

Certain embodiments of the subject matter described herein may be implemented in a computing system and/or devices that includes a back-end component (e.g., as a data server), or that includes a middleware component (e.g., an application server), or that includes a front-end component (e.g., a client computer having a graphical user interface or a Web browser through which a user may interact with an implementation of the subject matter described herein), or any combination of such back-end, middleware, or front-end components. The components of the system may be interconnected by any form or medium of digital data communication (e.g., a communication network). Examples of communication networks include a local area network (“LAN”), a wide area network (“WAN”), and the Internet.

The computing system according to some such embodiments described above may include clients and servers. A client and server are generally remote from each other and typically interact through a communication network. The relationship of client and server arises by virtue of computer programs running on the respective computers and having a client-server relationship to each other.

Any and all references to publications or other documents, including but not limited to, patents, patent applications, articles, webpages, books, etc., presented anywhere in the present application, are herein incorporated by reference in their entirety.

Example embodiments of the devices, systems and methods have been described herein. As noted elsewhere, these embodiments have been described for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Moreover, embodiments of the subject disclosure may include methods, systems and devices which may further include any and all elements from any other disclosed methods, systems, and devices, including any and all elements corresponding to translocation control. In other words, elements from one or another disclosed embodiments may be interchangeable with elements from other disclosed embodiments. In addition, one or more features/elements of disclosed embodiments may be removed and still result in patentable subject matter (and thus, resulting in yet more embodiments of the subject disclosure). Correspondingly, some embodiments of the present disclosure may be patentably distinct from one and/or another reference by specifically lacking one or more elements/features. In other words, claims to certain embodiments may contain negative limitation to specifically exclude one or more elements/features resulting in embodiments which are patentably distinct from the prior art which include such features/elements.

### REFERENCES

1. Branton, D., et al., Nanopore Sequencing. Nature Biotechnology, 2008. 26: p. 1146-1153.
2. Lu, B., F. Albertorio, D. P. Hoogerheide, and J. A. Golovchenko, Origins and Consequences of Velocity Fluctuations during DNA Passage through a Nanopore. Biophysical Journal, 2011. 101: p. 70-79.
3. Manrao, E. A., I. M. Derrington, A. H. Laszlo, K. W. Langford, M. K. Hopper, N. Gillgren, M. Pavlenok, M. Niederweis, and J. H. Gundlach, Reading DNA at single-nucleotide resolution with a mutant MspA nanopore and phi29 DNA polymerase. Nature Biotechnol., 2012. 30: p. 349-353.
4. Luan, B., H. Peng, S. Polonsky, S. Rossnagel, G. Stolovitzky, and G. Martyna, Base-By-Base Ratcheting of Single Stranded DNA through a Solid-State Nanopore. Phys. Rev. Lett., 2010. 104: p. 238103.
5. Polonsky, S., S. M. Rossnagel, and G. A. Stolovitzky, Systems and Methods for Controlling the Position of a Charged Polymer Inside a Nanopore, US Patent Number
6. Archakov, A. I.; Y. D. Ivanov, A. V. Lisitsa, and V. G. Zgoda, AFM fishing nanotechnology is the way to reverse the Avogadro number in proteomics. Proteomics, 2007. 7: p. 4-9.
7. Lindsay, S., J. He, O. Sankey, P. Hapala, P. Jelinek, P. Zhang, S. Chang, and S. Huang, Recognition Tunneling. Nanotechnology, 2010. 21: p. 262001-262013.
8. Huang, S., J. He, S. Chang, P. Zhang, F. Liang, S. Li, M. Tuchband, A. Fuhrman, R. Ros, and S. M. Lindsay, Identifying single bases in a DNA oligomer with electron tunneling. Nature Nanotechnology, 2010. 5: p. 868-73.
9. Liang, F., S. Li, S. Lindsay, and P. Zhang, Synthesis, Physicochemical Properties, and Hydrogen Bonding of 4(5)-Substituted-1H-imidazole-2-carboxamide, A Potential Universal Reader for DNA Sequencing by Recognition Tunneling. Chemistry—a. European Journal, 2012. 18: p. 5998-6007.
10. Fologea, D., J. Uplinger, B. Thomas, D. S. McNabb, and J. Li, Slowing DNA translocation in a solid-state nanopore. Nano Lett., 2005. 5: p. 1734.
11. Friddle, R., P. Podsiadlo, A. B. Artyukhin, and A. Noy, Near-Equilibrium Chemical Force Microscopy. 2008. J. Phys. Chem. C, 2008. 112: p. 4986-4990.
12. Friddle, R. W., A. Noy, and James J. De Yoreoa, Interpreting the widespread nonlinear force spectra of intermolecular bonds. Proc. Natl. Acad. Sci. (USA), 2012. 109: p. 13573-13578.
13. Keyser, U. F., B. N. Koelman, S. van Dorp, D. Krapf R. M. M. Smeets, S. G. Lemay, N. H. Dekker, and C. Dekker, Direct force measurements on DNA in a solid-state nanopore. Nature Physics, 2006. 2: p. 473-477.
14. Kimura-Suda, H., D. Y. Petrovykh, M. J. Tarlov, and L. J. Whitman, Base-Dependent Competitive Adsorption of Single-Stranded DNA on Gold. J. Am Chem Soc, 2003. 125: p. 9014-9015.
15. Chang, S., S. Sen, P. Zhang, B. Gyarfas, B. Ashcroft, S. Lefkowitz, H. Peng, and S. Lindsay, Palladium Electrodes for Molecular Tunnel Junctions. Nanotechnology, 2012. 23: p. in press.
16. Tomic, S., S. Dolanski-Babic, T. Vuletic, S. Krca, D. Invanovic, L. Griparic, and R. Podgornik, Dielectric relaxation of DNA aqueous solutions. Phys Rev E, 2007. 75: p. 21905-21918.
17. Sakamoto, M., T. Fujikado, R. Hayakawa, and Y. Wada, Low frequency dielectric relaxation and light scattering under ac electric field of DNA solutions. Biophysical Chemistry, 1980. 11: p. 309-316.
18. Sakamoto, M., R. Hayakawa, and Y. Wada, Dielectric relaxation of DNA solutions III. Effects of DNA concentration, protein contamination and mixed solvents. Biopolymers, 1979. 18: p. 2769-2782.
19. Sakamoto, M., H. Kanda, Y. Hyakawa, and Y. Wada, Dielectric relaxation of DNA in aqueous solutions. Biopolymers, 1976. 15: p. 879-892.
20. Chang, S., S. Huang, H. Liu, P. Zhang, R. Akahori, S. Li, B. Gyarfas, J. Shumway, B. Ashcroft, J. He, and S. Lindsay, Chemical Recognition and Binding Kinetics in a Functionalized Tunnel Junction. Nanotechnology, 2012.23: p. 235101-235115.
21. Keyser, U., Controlling molecular transport through nanopores. J. Roy. Soc. Interface, 2011. 8: p. 1369-1378
22. Wanunu, M., W. Morrison, Y. Rabin, A. Y. Grosberg, and A. Meller, Electrostatic focusing of unlabelled DNA into nanoscale pores using a salt gradient. Nature Nanotechnology, 2010. 5: p. 60-65.

1. A device for controlling the transit of a molecule across a nanopore comprising:
a first compartment;
a second compartment;
a first pair of electrodes comprising a first electrode provided in the first compartment and a second electrode provided in the second compartment;
a partition separating the first compartment from the second compartment;
an orifice provided in the partition;
a second pair of electrodes arranged proximate the orifice, the second pair of electrodes being functionalized with molecules; and
a tunnel gap comprising the spacing between the second pair of electrodes.

2. The device of claim 1, further comprising voltage bias configured to apply a voltage bias between the second pair of electrodes such that an electro-osmotic flow in a first direction for molecular transport is generated.

3. The device of claim 1, further comprising at least an AC voltage bias, wherein the AC voltage bias is configured to apply an AC voltage bias of at least 1 kHz in frequency between the second pair of electrodes.

4. The device of claim 3, further comprising processing means configured to process an AC current signal of the AC voltage bias in a non-linear mode such that upon the presence of a molecule in the tunnel gap can be detected.

5. The device of claim 1, further comprising a voltage bias configured to apply a voltage bias between at least one of the first electrode and the second pair of electrodes and the second electrode and the second pair of electrodes, and a circuit in communication with the second electrode pair, wherein the circuit is configured to control the voltage bias by a signal generated by the second electrode pair.

6. The device of claim 1, further comprising a voltage source configured to apply a voltage bias between the second electrode pair with both an AC and a DC component.

7-10. (canceled)

11. A method for controlling the transit of a molecule across a nanopore comprising:
providing a device for controlling the transit of a molecule across a nanopore, the device comprising:
a first compartment;
a second compartment;
a first pair of electrodes comprising a first electrode provided in the first compartment and a second electrode provided in the second compartment;
a partition separating the first compartment from the second compartment;
an orifice provided in the partition;
a second pair of electrodes arranged proximate the orifice, the second pair of electrodes being functionalized with molecules; and
a tunnel gap comprising the spacing between the second pair of electrodes; and
applying a voltage bias between the second pair of electrodes, wherein the voltage bias is configured to generate an electro-osmotic flow in a first direction for molecular transport.

12. The method of claim 11, further comprising applying an AC voltage of at least 1 kHz in frequency between the second pair of electrodes.

13. The method of claim 11, further comprising detecting the presence of a molecule in the tunnel gap via non-linear processing of the AC current signal.

14. The method of claim 11, further comprising applying a voltage bias between at least one of the first electrode and the second pair of electrodes and the second electrode and the second pair of electrodes, wherein said voltage bias is controlled by a circuit fed by a signal generated by the second electrode pair.

15. The method of claim 11, wherein the voltage bias comprises both an AC and a DC component.

16-17. (canceled)

18. A method for controlling translocation of uncharged molecules comprising:
providing a nanopore device for controlling translocation of uncharged molecules, the device comprising:
a first compartment;
a second compartment;
a first pair of electrodes comprising a first electrode provided in the first compartment and a second electrode providing in the second compartment;
a partition separating the first compartment from the second compartment;
a nanopore provided in the partition;
a second pair of electrodes arranged proximate the orifice, the second pair of electrodes being functionalized with molecules; and
a tunnel gap comprising the spacing between the second pair of electrodes, and
wherein the second pair of electrodes are biased so as to generate a Stokes flow into the nanopore; and
biasing the second pair of electrodes to generate a Stokes flow into the nanopore.

19. The device of claim 2, further comprising at least an AC voltage bias, wherein the AC voltage bias is configured to apply an AC voltage bias of at least 1 kHz in frequency between the second pair of electrodes.

20. The device of claim 19, further comprising processing means configured to process an AC current signal of the AC voltage bias in a non-linear mode such that upon the presence of a molecule in the tunnel gap can be detected.
